(19) United States
(12) Patent Application Publication
(10) Pub. No.: US 2004/0175338 A1
(43) Pub. Date: Sep. 9, 2004

(54) Cosmetic composition containing an ester and a pasty compound

(75) Inventors: Vanina Filippi, Paris (FR); Laure Le Chaux, Chevilly-Larue (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/792,801

(22) Filed: Mar. 5, 2004

Related U.S. Application Data
(60) Provisional application No. 60/455,863, filed on Mar. 20, 2003.
(60) Provisional application No. 60/455,866, filed on Mar. 20, 2003.

(30) Foreign Application Priority Data
Mar. 6, 2003 (FR) 03 02809
Mar. 6, 2003 (FR) 03 02811

(51) Int. Cl. A61K 7/025

(52) U.S. Cl. 424/64

(57) ABSTRACT

The present invention relates to a cosmetic composition for making up and/or caring for the skin, the lips and/or the integuments, comprising, in a physiologically acceptable medium, at least one ester of a diol dimer and of at least one C4 to C34 monocarboxylic acid or dicarboxylic acid, at least one pasty compound and at least one colouring agent, the said composition having a mean gloss of greater than or equal to 30.

### CROSS-REFERENCE TO RELATED APPLICATIONS

This nonprovisional application claims the benefit of U.S. Provisional Application No. 60/455,863, filed Mar. 20, 2003, and U.S. Provisional Application No. 60/455,866, filed Mar. 20, 2003.

### BACKGROUND OF THE INVENTION

1. Field of Invention

The present invention relates to a cosmetic composition for making up or caring for the skin, including the scalp, not only of the human face but also the body, and human lips or integuments, for instance the hair, the eyelashes, the eyebrows or the nails, comprising a cosmetically acceptable medium.

The composition of the invention may in particular be a makeup product for the human body, lips or integuments, in particular having non-therapeutic care and/or treatment properties. It is especially a lipstick, a lip gloss, a makeup rouge, an eyeshadow, a tattoo product, a mascara, an eyeliner, a nail varnish, a product for artificially tanning the skin, a hair-colouring product or a haircare product.

2. Description of Related Art

Many cosmetic compositions exist for which the gloss properties of the film deposited, after application to keratin materials (skin, lips or integuments), are desirable. Examples that may be mentioned include lipsticks, nail varnishes or certain hair products. To do this, the formulator generally uses, as main gloss active agent, lanolins combined with at least one “glossy” oil, for instance oily polymers such as a) polybutenes of high viscosity, b) fatty acid or fatty alcohol esters with a high carbon number (typically greater than 16), or c) certain plant oils.

### SUMMARY OF THE INVENTION

Oily polymers such as polybutenes have the drawback of being very sticky when applied and over time, which is unacceptable in terms of comfort for the user.

The object of the invention is, specifically, to propose a particular combination comprising at least one ester of a diol dimer with a monocarboxylic or dicarboxylic acid and in particular a specific dicarboxylic diacid, which is advantageously satisfactory in terms of gloss without otherwise affecting the comfort aspect of the cosmetic composition incorporating the said combination.

The compositions thus obtained present no particular stickiness problems and have no unpleasant odour.

Thus, lanolins have the drawback of being heat-sensitive and ultraviolet-sensitive and have a tendency to become oxidized over time, with release of an unpleasant odour. Furthermore, when lanolins are combined with oils commonly used in cosmetics, the compositions obtained have stickiness problems that are all the more pronounced when the oil used is of high viscosity.

This type of combination is all the more advantageous' since it makes it possible to dispense with the use of usual compounds such as lanolin and petroleum jelly, which, in certain circumstances, are no longer considered desirable, especially on account of a lack of stability, in cosmetic formulations.

Esters of diol dimers and of monocarboxylic or dicarboxylic acids have been described in general in document FR 2 795 309 as being useful for preparing cosmetic compositions especially having improved stability properties. More recently, documents JP 2002-128623, 2002-128628 and 2002-128629′ propose cosmetic compositions, especially makeup compositions, including as gloss active agent esters of dilinoleic diacids with dilinoleic diol dimers.

More specifically, according to one of its aspects, the present invention relates to a cosmetic composition for making up and/or caring for the skin, the lips and/or the integuments, comprising, in physiologically acceptable medium, at least one ester of a diol dimer and of at least one C₄-C₃₄ monocarboxylic acid or dicarboxylic acid, at least one pasty compound and at least one colouring agent, the composition having a mean gloss of greater than or equal to 30.

According to another of its aspects, the present invention relates to an anhydrous cosmetic composition for making up and/or caring for the skin, the lips and/or the integuments, comprising, in physiologically acceptable medium, at least one ester of a diol dimer and of a fatty acid or of a diacid dimer of a fatty acid, at least one pasty compound and less than 15% by weight of at least one filler.

The present invention also relates to a cosmetic composition for making up and/or caring for the skin, the lips and/or the integuments, comprising, in a physiologically acceptable medium, at least one ester of a diol dimer and of a diacid dimer of unsaturated fatty acids and at least one pasty compound.

The present invention also relates to a process for making up and/or caring for the skin, the lips and/or the integuments, comprising the application to the skin, the lips and/or the integuments of at least one composition according to the invention.

### DETAILED DESCRIPTION OF PREFERRED EMBODIMENTS

Gloss

In general, the cosmetic compositions and more particularly those intended to be applied to the lips may be characterized by a mean gloss index.

The term “mean gloss” means the gloss that may be measured using a conventional glossmeter, by the following method:

A coat 50 μm of the composition whose mean gloss it is desired to evaluate is spread onto a Leneta® brand contrast card of reference Form 1A Penopac, using an automatic spreader. The coat covers at least the white background of the card. The gloss at 20° is then measured on the white background using a Byk Gardner® glossmeter of reference microTri-Gloss.

The mean gloss of the composition is advantageously greater than or equal to 30, or even greater than or equal to 40, especially greater than or equal to 50, in particular greater than or equal to 60, more particularly greater than or equal to 65 or better still greater than or equal to 70, especially when the composition is intended to be applied to the lips.

For example, a cosmetic composition of lipstick type may have a mean gloss equal to about 60, a cosmetic composition of liquid gloss base type or a cosmetic composition of eyeshadow type may have a mean gloss equal to about 70, and a cosmetic composition of nail varnish base type may have a mean gloss equal to about 50.

The term “liquid gloss”, also known as liquid lipstick or lip gloss, denotes a fluid product intended to be applied to the lips and conditioned, for example, in a container provided with an applicator, this applicator comprising a handle member that also serves as the cap for closing the container, and an applicator member.

According to one variant of the invention, the comsmetic composition is in solid or cast form. For the purposes of the present invention, the term “solid” means any cosmetic composition lacking the ability to flow under the action of its own weight.

These compositions may, where appropriate, have a pasty appearance at room temperature (25° C.). Thus, a cosmetic composition may be in accordance with the invention from the moment that it has a melting point or a thermal transition temperature (for example a softening point) of greater than 25° C., which can range especially from 25 to 85° C., or even from 30 to 60° C. and in particular from 30 to 45° C. and/or a hardness that can range from 0.001 to 0.5 MPa and especially from 0.005 to 0.4 MPa.

The hardness of a composition may be determined by measuring the compression force, measured at 20° C. using a texturometer sold under the name TA-XT2i® by the company Rheo, equipped with a stainless-steel cylinder 2 mm in diameter travelling at a measuring speed of 0.1 mm/s, and penetrating into the composition to a penetration depth of 0.3 mm. The hardness value is the maximum compression force measured divided by the area of the texturometer cylinder in contact with the composition. In the particular case of lipsticks, the hardness may also be measured by the “cheese-wire” method, which consists in cutting a tube of lipstick 8.1 mm in diameter and measuring the hardness at 20° C., using a DFGHS 2® tensile testing machine from the company Indelco-Chatillon, travelling at a speed of 100 mm/minute. The hardness measured is expressed as the shear force (expressed in gram-force) required to cut a stick under these conditions. According to this method, the hardness of a stick composition according to the invention ranges especially from 50 to 300 g, preferably from 100 to 250 g, for example from 150 to 230 g. The highest values are generally preferred for compositions intended to be formulated as tubes with a diameter of 12.7 mm, and the lowest values for compositions intended for tubes 8.3 mm in diameter.

Ester of a Diol Dimer and of a Diacid Dimer of Unsaturated Fatty Acids

The diol dimer esters that may be used in the context of the present invention are commercially available or may be prepared in a conventional manner. They are especially of plant origin and may be obtained by esterification of a diol dimer with a C₄-C₃₄ monocarboxylic acid for instance a fatty acid, or with a dicarboxylic acid such as a diacid dimer.

In the case of esterification with a monocarboxylic acid, esters of relatively high molecular weight, ranging from about 1000 to 1300 g/mol, may be obtained; a diol dimer dicarboxylate with a weight-average molecular weight, determined by gel permeation chromatography (GPC), ranging from 2000 to 20 000 g/mol and preferably between 2000 and 4000 g/mol, may be obtained.

C₄-C₃₄ Monocarboxylic Acid

The monocarboxylic acid that may be used in the present invention contains from 4 to 34 carbon atoms and especially from 10 to 32 carbon atoms.

By way of illustration of monocarboxylic acids that are suitable for the invention, mention may be made especially of:

saturated linear acids such as butanoic acid, pentanoic acid, hexanoic acid, heptanoic acid, octanoic acid, nonanoic acid, decanoic acid, undecanoic acid, dodecanoic acid, tridecanoic acid, tetradecanoic acid, heptadecanoic acid, hexadecanoic acid, pentadecanoic acid, octadecanoic acid, nonadecanoic acid, eicosanoic acid, docosanoic acid and tetracosanoic acid,

branched fatty acids, for instance isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanbic acid, isotetradecanbic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyldodecanoic acid, 2-decyltetradecanoic acid, 2-dodecylhexadecanoic acid, 2-tetradecyloctadecanoic acid, 2-hexadecyloctadecanoic acid and long-chain fatty acids obtained from lanolin,

unsaturated linear C₈ to C₃₄ fatty acids, such as undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachidonic acid,

hydroxy acids such as 2-hydroxybutanoic acid, 2-hydropentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxy-tetradecanoic acid, 2-hydroxy-hexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid and 2-hydroxytetracosanoic acid,

cyclic acids such as cyclohexanoic acid, hydrogenated rosin, rosin, abietic acid, hydrogenated abietic acid, benzoic acid, p-oxybenzoic acid, p-aminobenzoic acid, cinnamic acid, p-methoxycinnamic acid, salicylic acid, gallic acid, pyrrolidonecarboxylic acid and nicotinic acid, and

fatty acids of natural origin, such as the fatty acids of orange oil, of avocado oil, of macadamia oil, of olive oil, of hydrogenated soybean oil, of jojoba oil, of palm oil, of castor oil, of wheatgerm oil, of saffron oil, of cottonseed oil and of mink oil, and mixtures thereof.

It is more particularly a fatty acid, especially as defined above.

The ester obtained may be a diester, a monoester or a mixture thereof. In the present case, the ester may be a mixture of two or more types of ester formed with different carboxylic acids.

Dicarboxylic Acid

The dicarboxylic acid that may be used according to the invention should contain at least two carboxylic groups per molecule.

It may be represented especially by formula (I) below:

HOOC—(CH₂)ₙ—COOH (I)

in which n is an integer from 1 to 16 and preferably from 3 to 16.

As non-limiting illustrations of dicarboxylic acids that are suitable for the invention, mention may be made especially of malonic acid, succinic acid, glutaric acid, adipic acid, pimelic acid, suberic acid, azelaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicarboxylic acid, 1,14-tetradecamethylenedicarboxylic acid, 1,15-pentadecamethylenedicarboxylic acid and 1,16-hexadecamethylenedicarboxylic acid, and mixtures thereof.

The dicarboxylic acid may also be a diacid dimer. The term “diacid dimer” denotes a diacid obtained by polymerization reaction, especially by intermolecular dimerization of at least one unsaturated fatty acid.

They are derived in particular from the dimerization of an unsaturated especially C₈ to C₃₄, especially C₁₂ to C₂₂, in particular C₁₆ to C₂₀ and more particularly C₁₈ fatty acid.

By way of representation of these unsaturated fatty acids, mention may be made especially, as previously, of undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachiconic acid, and mixtures thereof.

According to one particular variant, it is more particularly the diacid dimer from which the diol dimer to be esterified is also derived.

More particularly, it is the diacid dimer obtained by dimerization of linoleic acid and especially of its hydrogenated form. This hydrogenated form may be partial or total and may especially correspond to the saturated form, which is more stable to oxidation.

According to one embodiment, the diacid dimer is a commercial product consisting of a dicarboxylic acid containing about 36 carbon atoms. This product also contains a trimeric acid and a monomeric acid, in proportions that depend on the degree of purity of the product. Conventionally, products with a diacid dimer content of greater than 70% and others whose diacid dimer content has been adjusted to 90% or more are commercially available.

Diacid dimers and especially dilinoleic diacids whose stability towards oxidation has been improved by hydrogenation of the double bonds remaining after the dimerization reaction are also commercially available.

In the present invention, any diacid dimer that is currently commercially available may be used.

In an esterification reaction with a dicarboxylic acid and in particular a diacid dimer, the mean degree of esterification and the average molecular weight of the ester obtained may be adjusted by varying the ratio of the diol dimer to the dicarboxylic acid and in particular to the diacid dimer.

The ratio, expressed as the molar proportion of a dicarboxylic acid based on the average molecular weight calculated from its acid number per 1 mol of diol dimer based on the average molecular weight calculated from its hydroxyl number, is generally between 0.2 and 1.2 mol and in particular between 0.4 and 1.0, for example equal to 0.5 or 0.7.

Diol Dimer

For the purposes of the present invention, the term “diol dimer” is more particularly intended to denote saturated diols produced by hydrogenation of the corresponding diacid dimers, a diacid dimer being as defined above.

As regards the diol dimer manufacture industrially, it also generally contains other components, for example a triol trimer, a monalcohol and compounds of ether type, depending on the degree of purification of the dimeric acid and/or of the lower alcohol ester thereof, used as starting material. Generally, products whose diol dimer content is greater than 70% may be used in the present invention. However, it is preferable to use a diol dimer of high purity, such as a compound whose diol dimer content is greater than 90%.

Thus, a diol dimer may be produced by catalytic hydrogenation of a diacid dimer, which is itself obtained by dimerization of at least one unsaturated, especially C₈ to C₃₄ fatty acid, such as those mentioned above, especially C₁₂ to C₂₂, in particular C₁₆ to C₂₀ and more particularly C₁₈ fatty acid, such as, for example, oleic acid and linoleic acid.

According to one particular embodiment, the diol dimer is derived from the hydrogenation of dilinoleic diacid. It is generally in a saturated form.

According to one variant of the invention, the diol dimer ester is an ester of a diol dimer and of a diacid dimer and is especially a compound of general formula (II)

HO—R¹—(—OCO—R²—COO—R¹—)ₕ—OH (II)

in which:

R¹ represents a diol dimer residue obtained by hydrogenation of dilinoleic diacid

R² represents a hydrogenated dilinoleic diacid residue, and

h represents an integer ranging from 1 to 9.

By way of illustration of esters that are suitable for the invention, mention may be made especially of the esters of dilinoleic diacids and of dilinoleic diol dimers sold by the company Nippon Fine Cchemical under the trade name Lusplan DD-DA5® and DD-DA7®.

The amount of ester according to the invention is generally adjusted so as to control the mean gloss of the composition to the desired value. In the present case, the ester may be present in a proportion of from 1% to 99% in particular, especially from 2% to 60% by weight, in particular from 5% to 40% and more particularly from 10% to 35% by weight, relative to the total weight of the composition.

Pasty Compound

For the purposes of the present invention, the term “pasty” is intended to denote a lipophilic fatty compound other than lanolin and its derivatives, which undergoes a reversible solid/liquid change of state, having in the solid state an anisotropic crystal organization, and comprising at a temperature 23° C. a liquid fraction and a solid fraction. By way of illustration of the lanolin derivatives that are excluded from the pasty compounds according to the invention, mention may be made especially of liquid lanolin, reduced lanolin, adsorption-purified lanolin, lanolin acetate, lanolin wax, liquid lanolin acetate, hydroxylanolin, polyoxyethylene-lanolin, lanolin fatty acid, hard lanolin fatty acid, cholesteryl esters of lanolin fatty acid, lanolin alcohol, lanolin alcohol acetate, and the like.

For the purposes of the invention, the term “pasty compound” means a compound having a hardness at 20° C. ranging from 0.001 to 0.5 MPa and preferably from 0.002 to 0.4 MPa. The hardness is measured according to the protocol described previously, using a texture analyzer, for example the TA-XT2i® machine from Rheo.

This pasty compound is also, at a temperature of 23° C., in the form of a liquid fraction and a solid fraction. In other words, the starting melting point of the pasty compound is less than 23° C. The liquid fraction of the pasty compound measured at 23° C. represents 9% to 97% by weight of the compound. This liquid fraction at 23° C. preferably represents between 15% and 85% and more preferably between 40% and 85% by weight.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920′ by the company TA Instrument, with a temperature rise of 5 or 10° C. per minute, according to standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., consisting of a liquid fraction and a solid fraction.

The liquid fraction of the pasty compound, measured at 32° C., preferably represents from 30% to 100% by weight of the compound, preferably from 80% to 100% and more preferably from 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty compound is preferably chosen from synthetic compounds and compounds of plant origin. A pasty compound may be obtained by synthesis from starting materials of plant origin.

The pasty compound is advantageously chosen from:

polymer or non-polymer silicone compounds,

polymer or non-polymer fluoro compounds,

vinyl polymers, especially:

olefin homopolymers

olefin copolymers

hydrogenated diene homopolymers and copolymers

linear or branched oligomers, which are homopolymers or copolymers of alkyl (meth)acrylates preferably containing a C₈-C₃₀ alkyl group

oligomers, which are homopolymers and copolymers of vinyl esters containing C₈-C₃₀ alkyl groups

oligomers, which are homopolymers and copolymers of vinyl ethers containing C₈-C₃₀ alkyl groups,

liposoluble polyethers resulting from the polyetherification between one or more C₂-C₁₀₀ and preferably C₂-C₅₀ diols,

esters,

and mixtures thereof.

The pasty compound is preferably a polymer and especially a hydrocarbon-based polymer.

Silicone and/or Fluoro Pasty Compounds

A preferred silicone and fluoro pasty compound is polymethyl trifluoropropyl methylalkyl dimethylsiloxane, sold under the name X22-1088® by Shin-Etsu.

When the pasty compound is a silicone and/or fluoro polymer, the composition advantageously comprises a compatibilizer such as short-chain esters, for instance isodecyl neopentanoate.

Polyether Pasty Compounds

Among the liposoluble polyethers that are particularly preferred are copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30. In this family, mention will be made especially of copolymers such that the long-chain alkylene oxides are arranged in blocks with an average molecular weight of from 1000 to 10 000, for example a polyoxyethylene/polydodecyl glycol block copolymer such as the ethers of dodecanediol (22 mol) and of polyethylene glycol (45 EO) sold under the brand name Elfacos ST9® by Akzo Nobel.

Among the esters that are especially preferred are:

esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid and 12-hydroxystearic acid, for instance those sold under the brand name Softisan 649® by the company Sasol,

arachidyl propionate sold under the brand name Waxenol 801® by Akzo,

phytosteroi esters,

non-crosslinked polyesters resulting from polycondensation between a linear or branched C₄-C₅₀ dicarboxylic acid or polycarboxylic acid and a C₂-C₅₀ diol or polyol,

aliphatic esters of an ester resulting from the esterification of an aliphatic hydroxycarboxylic acid ester with an aliphatic carboxylic acid; and mixtures thereof.

The aliphatic carboxylic acid contains from 4 to 30′ and preferably from 8 to 30 carbon atoms. It is preferably chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoid acid, hexyldecanoic acid, heptadecanoic acid, octadecanoic acid, isostearic acid, nonadecanoic acid, eicosanoic acid, isoatachidic acid, octyldodecanoic acid, heneicosanoic acid and docosanoic acid, and mixtures thereof.

The aliphatic carboxylic acid is preferably branched.

The aliphatic hydroxycarboxylic acid ester is advantageously derived from a hydroxylated aliphatic carboxylic acid containing from 2 to 40 carbon atoms, preferably from 10 to 34 carbon atoms and better still from 12 to 28 carbon atoms, and from 1 to 20 hydroxyl groups, preferably from 1 to 10 hydroxyl groups and better still from 1 to 6 hydroxyl groups. The aliphatic hydroxycarboxylic acid ester is chosen from:

a) partial or total esters of saturated linear monohydroxylated aliphatic monocarboxylic acids;

b) partial or total esters of unsaturated monohydroxylated aliphatic monocarboxylic acids;

c) partial or total esters of saturated monohydroxylated aliphatic polycarboxylic acids;

d) partial or total esters of saturated polyhydroxylated aliphatic polycarboxylic acids;

e) partial or total esters of C₂ to C₁₆ aliphatic polyols that have reacted with a monohydroxylated or polyhydroxylated aliphatic monocarboxylic or polycarboxylic acid,

and mixtures thereof.

The aliphatic esters of an ester are advantageously chosen from:

the ester resulting from the esterification reaction of hydrogenated castor oil with isostearic acid in proportions of 1 to 1 (1/1) or hydrogenated castor oil monoisostearate,

the ester resulting from the esterification reaction of hydrogenated castor, oil with isostearic acid in proportions of 1 to 2 (1/2) or hydrogenated castor oil diisostearate,

the ester resulting from the, esterification reaction of hydrogenated castor oil with isotearic acid in proportions of 1 to 3 (1/3) or hydrogenated castor oil triisostearate,

and mixtures thereof.

Among the pasty compounds of plant origin that which will preferably be chosen is an oxyethylenated (5 eo) oxypropylenated (5 po) mixture of soybean sterols and of pentaerythritol, sold under the reference Lanolide® by the company Vevy.

The pasty compound preferably represents 1% to 99%, better still 1% to 60%, better still 2% to 30% and even better still 5% to 15% by weight of the composition.

In particular, the pasty compound and the ester may be combined in the cosmetic composition in a pasty compound/ester weight ratio ranging from 0.25 to 0.75 and especially from 0.3 to 0.6.

Physiologically Acceptable Medium

The term “physiologically acceptable medium” denotes a non-toxic medium that may be applied to human skin or lips. The physiologically acceptable medium is generally suited to the nature of the support onto which the composition is to be applied and also to the form in which the composition is intended to be packaged.

Aqueous Phase

The composition according to the invention may comprise at least one aqueous medium, constituting an aqueous phase, which may form the continuous phase of the composition.

The aqueous phase may consist essentially of water.

It may also comprise a mixture of water and of water-miscible organic solvent (miscible in water to greater than 50% by weight at 25° C.), for instance lower monoalcohols containing from 1 to 5 carbon atoms, such as ethanol, isopropanol, glycols containing from 2 to 8 carbon atoms, such as propylene glycol, ethylene glycol, 1,3-butylene glycol, dipropylene glycol, C₃-C₄ ketones and C₂-C₄ aldehydes.

The aqueous phase (water and optionally the water-miscible organic solvent) may be present in a content ranging from 1% to 95% by weight, especially ranging from 3% to 80% by weight, and in particular ranging from. 5% to 60% by weight, relative to the total weight of the composition.

This aqueous phase may, where appropriate, be thickened, gelled or structured by also incorporating therein a conventional aqueous-gelling agent especially of mineral origin, for instance clay, and/or of organic origin, for in stance an aqueous-gelling polymer.

Such a medium may also comprise at least one volatile oil as defined below.

Fatty Phase

The composition, especially when it is intended to be applied to the lips, may comprise a fatty phase and especially at least one, fatty substance that is liquid at room temperature (25° C.) and atomspheric pressure and/or a fatty substance that is solid at room temperature and atomspheric pressure, such as waxes and gums, and mixtures thereof. The fatty phase may also contain structuring and gelling agents of oils of organic nature and/or lipophilic organic solvents.

According to one variant, the cosmetic composition according to the invention may especially contain a continuous fatty phase, which may contain less than 5% water, especially less than 1% water, relative to its total weight, and may in particular be in anhydrous form.

According to one particular variant of the invention, the cosmetic composition is free of paraffin, of petroleum jelly, and, as mentioned previously, of lanolin or one of the derivatives thereof.

The fatty phase of the composition according to the invention may especially comprise, as liquid fatty substance, at least one volatile or non-volatile oil or a mixture thereof.

For the purposes of the invention, the term “volatile oil” means any oil capable of evaporating on contact with the skin in less than one hour, at room temperature and atmospheric pressure. The volatile oils of the invention are volatile cosmetic oils, which are liquid at room temperature, having a non-zero vapour pressure, at room temperature and atmospheric pressure, ranging in particular from 0.01 to 300 mmHg (1.33 Pa to 40 000 Pa) and preferably greater than 0.3 mmHg (30 Pa).

The term “non-volatile oil” means an oil that remains on the skin at room temperature and atmospheric pressure for at least several hours and that especially has a vapour pressure of less than 0.01 mmHg (1.33 Pa).

These volatile or non-volatile oils may be hydrocarbon-based oils, in particular of plant or animal origin, silicone oils or mixtures thereof. The term “hydrocarbon-based oil” means an oil mainly containing hydrogen and carbon atoms and possibly oxygen, nitrogen, sulphur and/or phosphorus atoms.

The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names Isopars® or Permetyls®, branched C₈-C₁₆ esters such isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The volatile oil may be present in the composition according to the invention in a content ranging from 0.1% to 98% by weight, especially from 1% to 65% by weight, and in particular from 2% to 50% by weight, relative to the total weight of the composition.

The non-volatile oils may be chosen especially from non-volatile, optionally fluoro, hydrocarbon-based oils and/or non-volatile silicone oils.

Non-volatile hydrocarbon-based oils that may especially be mentioned include:

hydrocarbon-based oils of animal origin,

hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stearineries Dubois or those sold under the names Miglyol 810®, 812® and 818® by the company Dynamit Nobel,

synthetic ethers containing from 10 to 40 carbon atoms;

linear or branched hydrocarbons of mineral or synthetic origin, such as petroleum jelly, polydecenes, hydrogenated polyisobutene such as parleam, and squalane, and mixtures thereof,

synthetic esters, for instance oils of formula R₃COOR₄ in which R₃ represents a linear or branched fatty acid residue containing from 1 to 40 carbon atoms and R₄ represents a hydrocarbon-based chain, which is especially branched, containing from 1 to 40 carbon atoms, on condition that R₃+R₄≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters,

fatty alcohols that are liquid at room temperature with a branched and/or unsaturated carbon-based chain containing from 0.12 to 26 carbon atoms, for instance octyldodecanol, isostearyl alcohol, oleyl alcohol, 2-hexyldecanol, 2-butyloctanol or 2-undecylpentadecanol,

higher fatty acids such as oleic acid, linoleic acid or linolenic acid, and mixtures thereof.

The non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each containing from 2 to 24 carbon atoms, phenylsilicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and mixtures thereof.

The non-volatile oils may be present in the composition according to the invention in a content ranging from 0.01% to 90% by weight, especially from 0.1% to 85% by weight and in particular from 1% to 70% by weight relative to the total weight of the composition.

The oils can represent from 0 to 99% of the total weight of the composition, in particular from 0.01% to 99%, more particularly from 0.05% to 60% and better still from 1% to 35%.

In the context of the present invention, oils with a molecular weight of between 650 and. 10 000 g/mol and especially between 750 and 7500 g/mol are used in particular.

According to one embodiment, the composition of the invention comprises an oily phase comprising at least 70% by weight of an oil with a molar mass of between 650 and 10 000 g/mol and preferably between 750 and 7500 g/mol. The oily phase advantageously comprises more than 80% and in particular more than 85% by weight of an oil with a molar mass of between 650 and 10 000 g/mol and more particularly between 750 and 7500 μg/mol.

The oil of high molar mass is preferably chosen from lipophilic polymers:

linear fatty acid esters with a total carbon number ranging from 35 to 70,

hydroxylated esters,

aromatic esters,

esters of a fatty alcohol or of fatty acids which are branched and of C₂₄-C₂₈,

silicone oils,

oils of plant origin,

and mixtures thereof.

The oil of high molar mass is preferably chosen from polybutylenes, hydrogenated polyisobutylenes, polydecenes, hydrogenated polydecenes, vinylpyrrolidone copolymers, such as the PVP/hexadecene copolymer, pentaerythrityl tetrapelargonate, polyglyceryl-2 triisostearate, tridecyl trimellitate, triisoarachidyl citrate, pentaerythrityl tetraisononanoate, glyceryl triisostearate, pentaerythrityl tetraisostearate, glyceryl tris(2-decyl)tetradecanoate, pentaerythrityl tetrakis(2-decyl)tetradecanoate, phenylsilicones and sesame oil, and mixtures thereof.

More generally, the fatty substance that is liquid at room temperature and atmospheric pressure may be present in a proportion of from 0.01% to 90% by weight and especially from 0.1% to 85% by weight relative to the weight of the fatty phase.

The cosmetic composition may comprise, especially when it is intended to be applied to the lips, an oily phase with a refractive index of between 1.47 and 1.51, which can allow a relatively high gloss to be obtained.

As regards the fatty substance that is solid at room temperature and atmospheric pressure, it may be chosen from waxes and gums and mixtures thereof. This solid fatty substance may be present in a proportion of from 0.01% to 50%, especially from 0.1% to 40% and in particular from 0.2% to 30% by weight relative to the total weight of the fatty phase.

Waxes and Gums

In particular, the composition may contain at least one wax.

For the purposes of the present invention, the term “wax” means a lipophilic fatty compound that is solid at room temperature (25° C.), which undergoes a reversible solid/liquid change of state, which has a melting point of greater than 30° C. which may be up to 200° C. and a hardness of greater than 0.5 MPa, and which has an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained.

The waxes that may be used in the invention are compounds that are solid at room temperature, intended to structure the composition, in particular in the form of a stick; they may be hydrocarbon-based waxes, fluoro waxes and/or silicone waxes and may be of plant, mineral, animal and/or synthetic origin. In particular, they have a melting point of greater than 40° C. and better still greater than 45° C.

As waxes that may be used in the invention, mention may be made of those generally used in cosmetics: they are especially of natural origin, for instance beeswax, carnauba wax, candelilla wax, ouricoury wax, Japan wax, cork fibre wax, sugarcane wax, rice wax, montan wax, paraffin, lignite wax or microcrystalline wax, ceresin, ozokerite and hydrogenated oils, for instance jojoba oil; synthetic waxes, for instance the polyethylene waxes derived from the polymerization or copolymerization of ethylene and Fischer-Tropsch waxes, or alternatively fatty acid esters, for instance octacosanyl stearate, glycerides that are solid at 40° C. and better still at 45° C., silicone waxes, for instance alkyl- or alkoxydimethicones containing an alkyl or alkoxy chain of 10 to 45 carbon atoms, poly(di)methylsiloxane esters that are solid at 40° C. and whose ester chain contains at least 10 carbon atoms; and mixtures thereof.

The gums that may be used in the invention are generally in dissolved form in an oil, the polymers are solid at room temperature and the resins may be liquid or solid at room temperature.

The term “gum” means a fatty substance that is in the form of a solid polymer at room temperature, with a weight-average molecular weight of from 50 000 to 1 000 000. The gum is often sold as a dispersion in an organic solvent such as silicone oil.

The nature and amount of the gums or waxes depend on the desired mechanical properties and textures. As a guide, the wax may represent from 0.01% to 50%, preferably from 2% to 40% and better still from 5% to 30% of the total weight of the composition.

In particular, the wax may be present in the form of a wax(es)-in-water emulsion.

The wax may be present in the composition according to the invention in a content ranging from 0.01% to 50% by weight, in particular from 0.1% to 30% by weight and especially from 0.2% to 20% by weight relative to the total weight of the composition.

Filler

For the purposes of the invention, this term is intended to denote any organic and/or mineral compound introduced into the cosmetic composition in order to adjust its texture properties or, in other words, to control its rheological properties. Pigments and nacres are especially excluded from this definition.

According to one particular variant of the invention, the cosmetic compositions comprise less than 15% by weight, in particular less than 10% by weight and especially less than 7% by weight of filler relative to the total weight of the composition.

They may especially be spherical fillers, for instance talc, zinc stearate, mica, kaolin, polyamide (Nylon®) (Orgasol® from Atochem) powders, polyethylene powders, tetrafluoroethylene polymer (Teflon®) powders, starch, boron nitride, polymer microspheres such as those of polyvinylidene chloride/acrylonitrile, for instance Expancel® (Nobel Industrie), acrylic acid copolymers (Polytrap® from the company Dow Corning), silicone resin microbeads (for example Tospearls® from Toshiba) and organopolysiloxane elastomers.

Surfactants

The composition according to the invention may also contain emulsifying surfactants present especially in a proportion ranging from 0.1% to 30% by weight and better still from 5% to 15% by weight relative to the total weight of the composition.

These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference, for the anionic and nonionic surfactants.

The surfactants preferably used in the composition according to the invention are chosen from:

nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated C₁-C₆ alkyl glucose fatty esters, and mixtures thereof,

anionic surfactants: C₁₆-C₃₀ fatty acids neutralized with amines, aqueous ammonia or alkaline salts, and mixtures thereof.

Surfactants that allow oil-in-water or wax-in-water emulsions to be obtained are preferably used.

Colouring Agent

The composition of the invention generally comprises at least one colouring agent, which may especially be present in a proportion of from 0.01% to 40% by weight, especially from 0.01% to 30% by weight and in particular from 0.05% to 25% by weight relative to the total weight of the composition.

This or these colouring agent(s) may be chosen from pigments, water-soluble or liposoluble dyes and nacres, and mixtures thereof.

The term “pigments” should be understood as meaning white or coloured, mineral or organic particles that are insoluble in the liquid hydrophilic phase, which are intended to colour and/or opacify the composition. The term “fillers” should be understood as meaning colourless or white, mineral or synthetic, lamellar or non-lamellar particles. The term “nacres” should be understood as meaning iridescent particles produced especially by certain molluscs in their shell, or alternatively synthesized.

The pigments may be present in the composition in a proportion of from 0.01% to 25% by weight, in particular from 0.01% to 15% by weight and especially from 0.02% to 5% by weight relative to the weight of the composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate. Among the organic pigments that may be used in the invention, mention may be made of carbon black, pigments of D & C type, and lakes based on cochineal carmine or on barium, strontium, calcium or aluminium, or alternatively the diketone pyrrolopyrroles (DPP) described in documents EP-A-542 669, EP-A-787 730, EP-A-787 731 and WO-A-96/08537. The amount and/or choice of these pigments is (are) generally adjusted by taking into account the amount of nanotubes present in the cosmetic composition under consideration.

The nacres may be present in the composition in a proportion of from 0.01% to 25% by weight, especially from 0.01% to 15% by weight and in particular from 0.02% to 5% by weight relative to the total weight of the composition.

The nacreous pigments may be chosen from white nacreous pigments such as mica coated with titanium or with bismuth oxychloride, coloured nacreous pigments such as titanium mica with iron oxides, titanium mica especially with ferric blue or with chromium oxide, titanium mica with an organic pigment of the abovementioned type and nacreous pigments based on bismuth oxychloride.

The composition may also comprise water-soluble or lipbsoluble dyes in a content ranging from 0.01% to 6% by weight and especially ranging from 0.01% to 3% by weight relative to the total weight of the composition. The liposoluble dyes are, for example, Sudan Red, DC Red 17, DC Green 6, β-carotene, soybean oil, Sudan Brown, DC Yellow 11, DC Violet 2, DC Orange 5 and quinoline yellow. The water-soluble dyes are, for example, beetroot juice and methylene blue.

The composition according to the invention may also comprise any ingredient conventionally used in the fields under consideration and more especially in cosmetics and dermatology. These ingredients are in particular chosen from vitamins, antioxidants, thickeners, trace elements, softeners, sequestering agents, fragrances, basifying or acidifying agents, preserving agents, UV-screening agents, hydrophilic or lipophilic active agents, and mixtures thereof. The amounts of these various ingredients are those conventionally used in the fields under consideration, for example from 0.01% to 20% of the total weight of the composition.

Needless to say, a person skilled in the art will take care to select this or these additional compound(s), and/or the amount thereof, such that the advantageous properties of the composition according to the invention are not, or are not substantially, adversely affected by the envisaged addition.

The composition of the invention may be obtained according to the preparation processes conventionally used in cosmetics or dermatology.

The composition of the invention may be in the form of a solid composition, compacted or cast in stick or dish form, or a pasty or liquid composition. It is advantageously in solid form, i.e. in hard form (which does not flow under its own weight) which has especially been cast or compacted, for example as a stick or a dish.

In the present case, it may be in the form of lipsticks, lip balms, cast foundations, concealer products, complexion “correctors” or “embellishers”, and/or eyeshadows or makeup rouges.

However, it may be in the form of a paste, a solid or a cream. It may be an oil-in-water or water-in-oil emulsion, a solid or supple anhydrous gel, or in the form of a free or compacted powder, and may even be in two-phase form. According to one particular variant, it is in the form of an emulsion.

The composition according to the invention may be in the form of a coloured or uncoloured composition, in the form of an antisun composition or makeup-removing composition or alternatively in the form of a hygiene composition. It especially contains cosmetic active agents. It may then be used as a care or treatment base for the skin, for instance the hands or the face, or for the lips (lip balms, for protecting the lips against the cold and/or sunlight and/or the wind) or alternatively as a deodorant. As cosmetic active agents that may be used in the invention, mention may be made of vitamins A, E, C and B3, provitamins, for instance D-panthenol, calmative active agents, for instance α-bisabolol, aloe vera and allantoin, plant extracts or essential oils, protective or restructuring agents, for instance ceramides, refreshing active agents, for instance menthol and derivatives thereof, emollients (cocoa butter, dimethicone), moisturizers (arginine PCA), anti-wrinkle active agents and essential fatty acids, and mixtures thereof.

The composition of the invention may also be in the form of a makeup product for the skin, in particular for facial skin, for instance a foundation, a blusher, a makeup such as a semi-permanent tattoo product or a lip makeup product, for instance a lipstick or lip gloss, optionally having care or treatment properties, a makeup product for the integuments, for instance a nail varnish, a mascara or an eyeliner, or a hair-colouring or haircare product.

Needless to say, the composition of the invention should be cosmetically acceptable, i.e. non-toxic and able to be applied to human skin, integuments or lips.

The examples of compositions below are given for illustrative purposes and without limiting nature.

### EXAMPLE 1

Two gloss formulations are prepared:

one, in accordance with the invention, incorporates the ester of dilinoleic acid and of dilinoleyl alcohol sold under the name Lusplan DD-DA7®, and

the other, a comparative formulation, replaces the said ester in equivalent amount with a polybutylene sold under the name Indopol H-1500® by the company Amoco.

Their respective compositions are as follows:

| | | Test 1 | Test 2 |
|---|---|---|---|
| Phase | Starting materials | (invention) | (comparative) |
| A | Diisostearyl malate | 50.9 | 50.9 |
| | Glyceryl ester of isostearic and | 9.5 | 9.5 |
| | adipic plant fatty acids (Softisan | | |
| | 649 ®) | | |
| | Preserving agent | qs | qs |
| | Mixture of isopropyl, isobutyl and | 0.29 | 0.9 |
| | n-butyl p-hydroxybenzoates | | |
| B | Polybutene (Indopol H-1500 ® | — | 30 |
| | from Amoco) | | |
| | Copolymers of dilinoleic diacids | 30 | — |
| | and of dilinoleic diols (Lusplan | | |
| | DD-DA7 from Nippon Fine | | |
| | Chemical) | | |
| C | Pigment | 2 | 2 |
| D | Silica | 0.3 | 0.3 |
| E | Fragrance | 7 | 7 |

The preparation protocol is as follows.

The pigment (phase C) is ground in 30 g of phase A. The ground material; the rest of phase A and phase B are then placed in a heating vessel heated to 70° C. and the mixture is homogenized for 30 minutes using a Rayneri, mixer. The silica (phase D) is then added to the mixture and the resulting mixture is homogenized for 2 hours. Finally, the fragrance is added to the mixture. This mixture is homogenized for 5 minutes and the stirring and heating are stopped. The mixture is allowed to cool to room temperature and the soft paste obtained is packaged in tubes.

It is noted that the formulation containing the dilinoleic diacid/dilinoleyl diol ester has better cosmetic properties than those containing the polybutylenes. Thus, the formulation with the dilonoleic diacid/dilinoleyl diol ester, Lusplan DD-DA7®, is less sticky, has a more slippery nature and is glossier when applied than that containing the polybutylene Indopol H-1500®. Furthermore, it is less sticky and glossier after application. After one hour, it is less sticky and has a more glossy nature.

### EXAMPLE 2

The above two formulations were reproduced, replacing in Test 1 the Lusplan DD-DA7® with Lusplan DD-DA5® and the Indopol H-1500® of Test 2 with Indopol H-100.

It is noted that the formulation in accordance with the invention is glossier after one hour than the control formulation.

### EXAMPLE 3

Two stick formulations are prepared:

one, in accordance with the invention, incorporates the ester of dilinoleic diacid and of dilinoleyl diol sold under the name Lusplan DD-DA5′, and

the other, a comparative formulation, replaces the said ester with an equivalent amount of liquid lanolin.

Their respective compositions are as follows:

| | | Test 3 | Test 4 |
|---|---|---|---|
| Phase | Starting materials | (invention) | (comparative) |
| A | Sesame oil | 24 | 24 |
| | Preserving agent | qs | qs |
| | Dilinoleic diacids/dilinoleyl diols | 16.1 | — |
| | copolymers (Lusplan DD-DA5 ® | | |
| | from Nippon Fine Chemical) | | |
| | Liquid lanolin | — | 16.1 |
| | Glyceryl esters of isostearic and | 8 | 8 |
| | adipic plant fatty acids | | |
| B | Sesame oil | 12.1 | 12.1 |
| | Hectorite modified with distearyl | 0.6 | 0.6 |
| | dimethylammonium chloride | | |
| | Dilinoleyl diol dimers/dilinoleic | 8.1 | 8.1 |
| | dimers copolymers (Lusplan | | |
| | DD-DA5 ®) | | |
| | Liquid lanolin | — | — |
| | Glyceryl esters of isostearic and | 4.1 | 4.1 |
| | adipic plant fatty acids (Softisan | | |
| | 649 ®) | | |
| C | Microcrystalline wax (Microwax | 2.5 | 2.5 |
| | HW ® from Paramelt) | | |
| | Polyglyerolated beeswax | 4.2 | 4.2 |
| | Microcrystalline wax (Base Wax | 8 | 8 |
| | 30540 ® from Paramelt) | | |
| D | Titanium dioxide | 1.8 | 1.8 |
| | Pigments | 6.9 | 6.9 |
| E | Active agents | 3.6 | 3.6 |

The preparation protocol is as follows:

The bentone gel (phase B) is prepared using a high-pressure homogenizer. The pigments (phase D) are ground in phase A. The whole is then placed in a heating vessel with phase C, heated to 100° C. and homogenized using a Rayneri mixer for two hours. 5 minutes before casting, the active phase E is added. The mixture is then cast in moulds at 42° C. and the moulds are placed in a freezer at −20° C. for half an hour, after which the sticks are stripped therefrom.

It is noted that, when applied to the lips, the formulation containing the dilinoleic acid/dilinoleyl diol ester is glossier at 1 hour than that containing the liquid lanolin, all the other properties being otherwise identical.

What is claimed is:

1. A cosmetic composition for making up and/or caring for the skin, the lips and/or the integuments, comprising, in physiologically acceptable medium, at least one ester of a diol dimer and of at least one C₄-C₃₄ monocarboxylic acid or dicarboxylic acid, at least one pasty compound and at least one colouring agent, said composition having a mean gloss of greater than or equal to 30.

2. An anhydrous cosmetic composition for making up and/or caring for the skin, the lips and/or the integuments, comprising, in a physiologically acceptable medium, at least one ester of a diol dimer and of a fatty acid or a diacid dimer of a fatty acid, at least one pasty compound and less than 15% by weight of at least one filler.

3. A cosmetic composition for making up and/or caring for the skin, the lips and/or the integuments, comprising, in a physiologically acceptable medium, at least one ester of a diol dimer and of a diacid dimer of unsaturated fatty acids, and at least one pasty compound.

4. The composition according to claim 1, characterized in, that the monocarboxylic acid is selected from the group consisting of:
saturated linear acids such as butanoic acid, pentanoic acid, hexanoic acid, heptanoic acid, octanoic acid, nonanoic acid, decanoic acid, undecanoic acid, dodecanoic acid, tridecanoic acid, tetradecanoic acid, heptadecanoic acid, hexadecanoic acid, pentadecanoic acid, octadecanoic acid, nonadecanoic acid, eicosanoic acid, docosanoic acid and tetracosanoic acid,
fatty acids,
hydroxy acids such as 2-hydroxybutanoic acid, 2-hydropentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxy-tetradecanoic acid, 2-hydroxy-hexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid and 2-hydroxytetracosanoic acid,
cyclic acids such as cyclohexanoic acid, hydrogenated rosin, rosin, abietic acid, hydrogenated abietic acid, benzoic acid, p-oxybenzoic acid, p-aminobenzoic acid, cinnamic acid, p-methoxycinnamic acid, salicylic acid, gallic acid, pyrrolidonecarboxylic acid and nicotinic acid, and
mixtures thereof.

5. The composition according to claim 2, characterized in that the fatty acid is selected from the group consisting of:
branched fatty acids, for instance isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isodecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyldodecanoic acid, 2-decyltetradecanoic acid, 2-dodecylhexadecanoic acid, 2-tetradecyloctadecanoic acid and 2-hexadecyloctadecanoic acid,
unsaturated linear C₈ to C₃₄ fatty acids, such as undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachidonic acid,
fatty acids of natural origin, such as the fatty acids of orange oil, of avocado oil, of macadamia oil, of olive oil, of hydrogenated soybean oil, of jojoba oil, of palm oil, of castor oil, of wheatgerm oil, of saffron oil, of cottonseed oil and of mink oil, and
mixtures thereof.

6. The composition according to claim 1, characterized in that the dicarboxylic acid is selected from the group consisting of:
the compounds of formula (I) below:
HOOC—(CH₂)ₙ—COOH (I)
in which n is an integer from 1 to 16 and preferably from 3 to 16, and
the diacid dimers obtained by intermolecular dimerization of at least one unsaturated fatty acid.

7. The composition according to claim 6, characterized in that the dicarboxylic acid is a diacid dimer.

8. The composition according to claim 3, characterized in that the unsaturated fatty acid is an unsaturated C₈ to C₃₄, especially C₁₂- to C₂₂, in particular C₁₆ to C₂₀ and more particularly C₁₈ fatty acid.

9. The composition according to claim 3, characterized in that the unsaturated fatty acid is selected from the group consisting of undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, arachidonic acid, and mixtures thereof.

10. The composition according to claim 2, characterized in that the diacid dimer is derived from the dimerization of linoleic acid.

11. The composition according to claim 2, characterized in that the diacid dimer is in its hydrogenated form.

12. The composition according to claim 1, characterized in that the diol dimer is derived from the hydrogenation of a diacid dimer.

13. The composition according to claim 12, characterized in that the diacid dimer is derived from the dimerization of an unsaturated, especially C₈ to C₃₄, in particular C₁₂ to C₂₂, in particular C₁₆ to C₂₀ and more particularly C₁₈ fatty acid.

14. The composition according to claim 13, characterized in that the unsaturated fatty acid is an unsaturated linear C₈ to C₃₄ fatty acid, such as undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachidonic acid, and mixtures thereof.

15. The composition according to claim 1, characterized in that the diol dimer is derived from the hydrogenation of dilinoleic diacid.

16. The composition according to claim 1, characterized in that the diol dimer is in a saturated form.

17. The composition according to claim 12, characterized in that the diacid dimer is identical to the diacid dimer from which the diol dimer is derived.

18. The composition-according to claim 1, characterized in that the diol dimer ester is an ester of a diol dimer and of a diacid dimer.

19. The composition according to claim 1, characterized in that the ester is a compound of general formula (II):
HO—R¹—(—OCO—R²—COO—R¹—)ₕ—OH
in which:
R¹ represents a diol dimer residue obtained by hydrogenation of dilinoleic diacid,
R² represents a hydrogenated dilinoleic diacid residue, and
h represents an integer ranging from 1 to 9.

20. The composition according to claim 1, characterized in that the ester is present in a proportion of from 1% to 99% by weight, especially from 2% to 60% by weight, in particular from 5% to 40% by weight and more particularly from 10% to 35% by weight, relative to the total weight of the composition.

21. The composition according to claim 1, characterized in that the composition has a mean gloss of greater than or equal to 60 and in particular greater than or equal to 70.

22. The composition according to claim 1, characterized in that the pasty compound has a hardness at 20° C. ranging from 0.001 to 0.5 MPa and especially from 0.002 to 0.4 MPa.

23. The composition according to claim 1, characterized in that the pasty compound has an anisotropic crystal organization in the solid state and comprises, at a temperature of 23° C., a liquid fraction and a solid fraction.

24. The composition according to claim 23, characterized in that the liquid fraction at 23° C. represents between 15% and 85% and in particular between 40% and 85% by weight.

25. The composition according to claim 23, characterized in that the liquid fraction of the pasty compound, measured at 32° C., represents from 30% to 100% by weight of the compound, in particular from 80% to 100% and more particularly from 90% to 100% by weight of the compound.

26. The composition according to claim 1, characterized in that the pasty compound is selected from the group consisting of:
polymer or non-polymer silicone compounds,
polymer or non-polymer fluoro compounds,
vinyl polymers, especially:
olefin homopolymers
olefin copolymers
hydrogenated diene homopolymers and copolymers
linear or branched oligomers, which are homopolymers or copolymers of alkyl (meth)acrylates preferably containing a C₈-C₃₀ alkyl group
oligomers, which are homopolymers and copolymers of vinyl esters containing C₈-C₃₀ alkyl groups
oligomers, homopolymers and copolymers of vinyl ethers containing C₈-C₃₀ alkyl groups,
liposoluble polyethers resulting from the polyetherification between one or more C₂-C₁₀₀ and preferably C₂-C₅₀ diols,
esters,
and mixtures thereof.

27. The composition according to claim 1, characterized in that the pasty compound is hydrocarbon-based.

28. The composition according to claim 1, characterized in that the pasty compound is polymethyl trifluoropropyl methylalkyl dimethylsiloxane.

29. The composition according to claim 26, characterized in that the liposoluble polyether is chosen from copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30.

30. The composition according to claim 29, characterized in that the liposoluble polyether is a polyoxyethylene/polydodecyl glycol block copolymer.

31. The composition according to claim 26, characterized in that the esters are selected from the group consisting of:
esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid and 12-hydroxystearic acid,
arachidyl propionate,
phytosterol esters,
non-crosslinked polyesters resulting from polycondensation between a linear or branched C₄-C₅₀ dicarboxylic acid or polycarboxylic acid and a C₂′-C₅₀ diol or polyol,
aliphatic esters of an ester resulting from the esterification of an aliphatic hydroxycarboxylic acid ester with an aliphatic carboxylic acid; and
mixtures thereof.

32. The composition according to claim 31, characterized in that the carboxylic acid is selected from the group consisting of hexanoic acid, heptanoic acid, octanoic acid, 2-ethyl-hexanoic acid, nonanoic acid; decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid, and mixtures thereof.

33. The composition according to claim 31, characterized in that the aliphatic hydroxycarboxylic acid ester is selected from the group consisting of:
a) partial or total esters of saturated linear monohydroxylated aliphatic monocarboxylic acids;
b) partial or total esters of unsaturated monohydroxylated aliphatic monocarboxylic acids;
c) partial or total esters of saturated monohydroxylated aliphatic polycarboxylic acids;
d) partial or total esters of saturated polyhydroxylated aliphatic polycarboxylic acids;
e) partial or total esters of C₂ to C₁₆ aliphatic polyols that have reacted with a monohydroxylated or polyhydroxylated aliphatic monocarboxylic or polycarboxylic acid,
and mixtures thereof.

34. The composition according to claim 1, characterized in that the pasty compound is present in a proportion of from 1% to 99% by weight, especially from 1% to 60% by weight, in particular from 2% to 30% by weight and more particularly from 5% to 15% by weight relative to the total weight of the composition.

35. The composition according to claim 1, characterized in that the composition has a melting point or a thermal transition temperature of greater than 25° C., especially ranging from 25 to 85° C. and in particular ranging from 30 to 60° C., and/or a hardness ranging from 0.001 to 0.5 MPa and especially ranging from 0.005 to 0.4 MPa.

36. The composition according to ° claim 3, characterized in that the composition comprises at least one aqueous phase.

37. The composition according to claim 1, characterized in that the composition comprises at least one fatty phase.

38. The composition according to claim 1, characterized in that the composition is anhydrous.

39. The composition according to claim 37, characterized in that said fatty phase contains at least one fatty substance that is liquid at room temperature and atmospheric pressure and/or at least one fatty substance that is solid at room temperature and atmospheric pressure.

40. The composition according to claim 39, characterized in that the said fatty substance that is liquid at room temperature and atmospheric pressure comprises at least one volatile or non-volatile oil or a mixture thereof.

41. The composition according to claim 40, characterized in that the non-volatile oil is selected from the group consisting of hydrocarbon-based oils of animal origin; hydrocarbon-based plant oils; linear or branched hydrocarbons of mineral or synthetic origin; synthetic ethers containing from 10 to 40 carbon atoms; synthetic esters, polyol esters; fatty alcohols containing from 12 to 26 carbon atoms; higher fatty acids; silicone oils of polymethylsiloxane (PDMS) type, and mixtures thereof.

42. The composition according to claim 39, characterized in that the liquid fatty substance comprises at least one volatile oil chosen from hydrocarbon-based oils containing from 8 to 16 carbon atoms and volatile silicone oils.

43. The composition according to claim 39, characterized in that said fatty substance that is liquid at room temperature and atmospheric pressure represents from 0.01% to 90% and especially from 0.1% to 85% by weight relative to the total weight of the fatty phase.

44. The composition according to claim 39, characterized in that said fatty substance that is solid at room temperature and atmospheric pressure is chosen from waxes and gums, and mixtures thereof.

45. The composition according to claim 39, characterized in that said fatty phase contains at least one solid fatty substance in a proportion of from 0.01% to 50%, especially from 0.1% to 40% and in particular from 0.2% to 30% by weight relative to the total weight of the fatty phase.

46. The composition according to claim 1, characterized in that the composition comprises less than 15% by weight, in particular less than 10% by weight and especially less than 7% by weight of at least one filler relative to its total weight.

47. The composition according to claim 46, characterized in that the filler is chosen from spherical fillers, for instance talc, zinc stearate, mica, kaolin, polyamide powders, polyethylene powders, tetrafluoroethylene polymer powders, starch, boron nitride, polymer microspheres such as those of polyvinylidene chloride/acrylonitrile, acrylic acid copolymers, silicone resin microbeads and organopolysiloxanes.

48. The composition according to claim 1, characterized in that said composition comprises said at least one colouring agent in a proportion of from 0.01% to 40%, especially from 0.1% to 30% and in particular from 0.2% to 25% by weight relative to the total weight of the composition.

49. The composition according to claim 48, characterized in that said colouring agent is selected from the group consisting of pigments, liposoluble or water-soluble dyes, nacres, and mixtures thereof.

50. The composition according to claim 1, characterized in that the composition is in the form of an emulsion.

51. The composition according to claim 1, characterized in that the composition is in the form of products cast as a stick or a dish.

52. The composition according to claim 1, characterized in that the composition is in the form of lipsticks, lip balms, cast foundations, concealer products, complexion “correctors” or “embellishers”, and/or eyeshadows or makeup rouges.

53. A process for making up and/or caring for skin, lips, or integuments, comprising applying to the skin, the lips and/or the integuments at least one composition according to claim 1.

54. The composition according to claim 4, characterized in that the fatty acid is selected from the group consisting of:
branched fatty acids, for instance isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyldodecanoic acid, 2-decyltetradecanoic acid, 2-dodecylhexadecanoic acid, 2-tetradecyloctadecanoic acid and 2-hexadecyloctadecanoic acid,
unsaturated linear C₈ to C₃₄ fatty acids, such as undecenoic acid, linderic acid, myristoleic acid, palmitoleic acid, oleic, acid, linoleic acid, elaidinic acid, gadolenoic acid, eicosapentaenoic acid, docosahexaenoic acid, erucic acid, brassidic acid and arachidonic acid,
fatty acids of natural origin, such as the fatty acids of orange oil, of avocado oil, of macadamia oil, of olive oil, of hydrogenated soybean oil, of jojoba oil, of palm oil, of castor oil, of wheatgerm oil, of saffron oil, of cottonseed oil and of mink oil, and
mixtures thereof.

55. The composition according to claim 5, characterized in that the unsaturated fatty acid is an unsaturated C₈ to C₃₄, especially C₁₂ to C₂₂, in particular C₁₆ to C₂₀ and more particularly C₁₈ fatty acid.

56. The composition according to claim 3, characterized in that the diacid dimer is in its hydrogenated form.

57. The composition according to claim 7, characterized in that the diacid dimer is in its hydrogenated form.

58. The composition according to claim 2, characterized in that the diol dimer is in a saturated form.

59. The composition according to claim 3, characterized in that the diol dimer is in a saturated form.

60. The composition according to claim 2, characterized in that the diol dimer ester is an ester of a diol dimer and of a diacid dimer.

61. The composition according to claim 3, characterized in that the diol dimer ester is an ester of a diol dimer and of a diacid dimer.

62. The composition according to claim 2, characterized in that the ester is a compound of general formula (II):
HO—R¹—(—OCO—R²—COO—R¹—)ₕ—OH
in which:
R¹ represents a diol dimer residue obtained by hydrogenation of dilinoleic diacid,
R² represents a hydrogenated dilinoleic diacid residue, and
h represents an integer ranging from 1 to 9.

63. The composition according to claim 3, characterized in that the ester is a compound of general formula (II):
HO—R¹—(—OCO—R²—COO—R¹—)ₕ—OH
in which:
R¹ represents a diol dimer residue obtained by hydrogenation of dilinoleic diacid,
R² represents a hydrogenated dilinoleic diacid residue, and
h represents an integer ranging from 1 to 9.

64. The composition according to claim 2, characterized in that the ester is present in a proportion of from 1% to 99% by weight, especially from 2% to 60% by weight, in particular from 5% to 40% by weight and more particularly from 10% to 35% by weight, relative to the total weight of the composition.

65. The composition according to claim 3, characterized in that the ester is present in a proportion of from 1% to 99% by weight, especially from 2% to 60% by weight, in particular from 5% to 40% by weight and more particularly from 10% to 35% by weight, relative to the total weight of the composition.

66. The composition according to claim 2, characterized in that the composition has a mean gloss of greater than or equal to 30, especially greater than or equal to 60 and in particular greater than or equal to 70.

67. The composition according to claim 3, characterized in that the composition has a mean gloss of greater than or equal to 30, especially greater than or equal to 60 and in particular greater than or equal to 70.

68. The composition according to claim 2, characterized in that the pasty compound has a hardness at 20° C. ranging from 0.001 to 0.5 MPa and especially from 0.002 to 0.4 MPa.

69. The composition according to claim 3, characterized in that the pasty compound has a hardness at 20° C. ranging from 0.001 to 0.5 MPa and especially from 0.002 to 0.4 MPa.

70. The composition according to claim 2, characterized in that the pasty compound has an anisotropic crystal organization in the solid state and comprises, at a temperature of 23° C., a liquid fraction and a solid fraction.

71. The composition according to claim 3, characterized in that the pasty compound has an anisotropic crystal organization in the solid state and comprises, at a temperature of 23° C., a liquid fraction and a solid fraction.

72. The composition according to claim 2, characterized in that the pasty compound is selected from the group consisting of:
polymer or non-polymer silicone compounds,
polymer or non-polymer fluoro compounds,
vinyl polymers, especially:
olefin homopolymers
olefin copolymers
hydrogenated diene homopolymers and copolymers
linear or branched oligomers, which are homopolymers or copolymers of alkyl (meth)acrylates preferably containing a C₈-C₃₀ alkyl group
oligomers, which are homopolymers and copolymers of vinyl esters containing C₈-C₃₀ alkyl groups
oligomers, homopolymers and copolymers of vinyl ethers containing C₈-C₃₀ alkyl groups,
liposoluble polyethers resulting from the polyetherification between one or more C₂-C₁₀₀ and preferably C₂-C₅₀ diols,
esters,
and mixtures thereof.

73. The composition according to claim 3, characterized in that the pasty compound is selected from the group consisting of:
polymer or non-polymer silicone compounds,
polymer or non-polymer fluoro compounds,
vinyl polymers, especially:
olefin homopolymers
olefin copolymers
hydrogenated diene homopolymers and copolymers
linear or branched oligomers, which are homopolymers or copolymers of alkyl (meth)acrylates preferably containing a C₈-C₃₀ alkyl group
oligomers, which are homopolymers and copolymers of vinyl esters containing C₈-C₃₀ alkyl groups
oligomers, homopolymers and copolymers of vinyl ethers containing C₈-C₃₀ alkyl groups,
liposoluble polyethers resulting from the polyetherification between one or more C₂-C₁₀ and preferably C₂-C₅₀ diols,
esters,
and mixtures thereof.

74. The composition according to claim 2, characterized in that the pasty compound is hydrocarbon-based.

75. The composition according to claim 3, characterized in that the pasty compound is hydrocarbon-based.

76. The composition according to claim 2, characterized in that the pasty compound is polymethyl trifluoropropyl methylalkyl dimethylsiloxane.

77. The composition according to claim 3, characterized in that the pasty compound is polymethyl trifluoropropyl methylalkyl dimethylsiloxane.

78. The composition according to claim 2, characterized in that the pasty compound is present in a proportion of from 1% to 99% by weight, especially from 1% to 60% by weight, in particular from 2% to 30% by weight and more particularly from 5% to 15% by weight relative to the total weight of the composition.

79. The composition according to claim 3, characterized in that the pasty compound is present in a proportion of from 1% to 99% by weight, especially from 1% to 60% by weight, in particular from 2% to 30% by weight and more particularly from 5% to 15% by weight relative to the total weight of the composition.

80. The composition according to claim 2, characterized in that the composition has a melting point or a thermal transition temperature of greater than 25° C., especially ranging from 25 to 85° C. and in particular ranging from 30 to 60° C., and/or a hardness ranging from 0.001 to 0.5 MPa and especially ranging from 0.005 to 0.4 MPa.

81. The composition according to claim 3, characterized in that the composition has a melting point or a thermal transition temperature of greater than 25° C., especially ranging from 25 to 85° C. and in particular ranging from 30 to 60° C., and/or a hardness ranging from 0.001 to 0.5 MPa and especially ranging from 0.005 to 0.4 MPa.

82. The composition according to claim 1, characterized in that the composition comprises at least one aqueous phase.

83. The composition according to claim 2, characterized in that the composition comprises at least one fatty phase.

84. The composition according to claim 3, characterized in that the composition comprises at least one fatty phase.

85. The composition according to claim 3, characterized in that the composition is anhydrous.

86. The composition according to claim 2, characterized in that the composition comprises less than 10% by weight and especially less than 7% by weight of at least one filler relative to its total weight.

87. The composition according to claim 3, characterized in that the composition comprises less than 15% by weight, in particular less than 10% by weight and especially less than 7% by weight of at least one filler relative to its total weight.

88. The composition according to claim 2, characterized in that said composition also comprises at least one colouring agent, especially in a proportion of from 0.01% to 40%, especially from 0.1% to 30% and in particular from 0.2% to 25% by weight relative to the total weight of the composition.

89. The composition according to claim 3, characterized in that said composition also comprises at least one colouring agent, especially in a proportion of from 0.01% to 40%, especially from 0.1% to 30% and in particular from 0.2% to 25% by weight relative to the total weight of the composition.

90. The composition according to claim 3, characterized in that the composition is in the form of an emulsion.

91. A process for making up and/or caring for skin, lips, or integuments, comprising applying to the skin, the lips and/or the integuments at least one composition according to claim 2.

92. A process for making up and/or caring for skin, lips, or integuments, comprising applying to the skin, the lips and/or the integuments at least one composition according to claim 3.
